(19) United States
(12) Patent Application Publication
(10) Pub. No.: US 2026/0151272 A1
(43) Pub. Date: Jun. 4, 2026

(54) BANDAGE WITH PEELABLE COVER

(71) Applicant: CLEAR CARE PRODUCTS, INC., Alachua, FL (US)

(72) Inventors: Karl Robert Zawoy, High Springs, FL (US); Stephen Frank Larner, Newberry, FL (US); Susan Powers, Gainesville, FL (US)

(21) Appl. No.: 19/375,227

(22) Filed: Oct. 31, 2025

Related U.S. Application Data
(60) Provisional application No. 63/727,171, filed on Dec. 2, 2024.

(51) Int. Cl. A61F 13/0203; A61F 13/00

(52) U.S. Cl. CPC A61F 13/0203; A61F 13/00063; A61F 2013/00119; A61F 2013/0017; A61F 2013/00182; A61F 2013/0057

(57) ABSTRACT

The present invention relates to a peelable wound dressing system configured to maintain a controlled wound environment while enabling repeated access for treatment. The system includes a flexible base layer adhered to skin and a transparent peelable cover affixed by a resealable attachment mechanism. The cover is configured for multiple peel-and-reseal cycles, permitting wound inspection and treatment without removing the base layer. Optional features include antimicrobial agents in the base layer, anti-fog treatments in the cover, perforations or catheters for exudate drainage, and access pathways for lavage, infusion, or drug delivery. In some embodiments, sensor networks and wireless communication circuits are integrated into the cover or base to monitor wound and skin parameters, with power supplied by micro-batteries, passive RFID/NFC, or energy-harvesting elements. These features collectively improve infection control, visualization, treatment access, monitoring, and extended wear, addressing critical unmet needs in human and veterinary wound care.

### CROSS-REFERENCE TO RELATED APPLICATIONS

The present application claims priority to U.S. Provisional patent application No. 63/727,171 filed on Dec. 2, 2024, the contents of the foregoing is herein incorporated by reference.

### FIELD OF INVENTION

The present invention relates generally to wound care products and devices and, more particularly, to transparent wound care products that incorporate absorptive materials, non-invasive sensors, and treatment pathways enabling monitoring and application of medicaments.

### BACKGROUND OF THE INVENTION

The healthcare industry continues to evolve to address patient needs, particularly in wound care management, which remains a critical component of treatment for both humans and animals. Wound care presents persistent challenges in cases involving chronic wounds, extensive injuries, and environments requiring frequent dressing changes. Although advanced bandages and dressings have been developed, unmet needs remain in balancing ease of use, affordability, flexibility, durability, and hygienic maintenance, all of which are essential for wound recovery.

The application of bandages has progressed from basic cloth wrappings to sophisticated materials designed to promote healing and prevent infection. Despite these advancements, the global wound care industry continues to encounter significant challenges in managing the complexity of wounds. For example, in veterinary wound care, the ability to frequently inspect and treat wounds without disturbing the dressing is a critical need, yet many existing products do not adequately meet this requirement. In many cases, removing and reapplying bandages causes additional trauma, prolongs healing, and increases the risk of infection due to exposure of the wound to infectious agents during bandage changes.

Similarly, in human wound care, chronic conditions such as diabetes often result in wounds that require constant monitoring and intervention. Traditional dressings provide basic protection but frequently require removal, creating risks of contamination, tearing of the patient's skin, reopening of the wound, and complications in the healing process. These limitations reveal a critical unmet need for bandages that provide both protection and visibility of the wound without compromising the healing environment.

One of the most pressing challenges in wound care is providing visual access to the wound without compromising protection. In traditional bandaging systems, frequent removal is necessary for inspection, which increases the risk of exposing the wound to contaminants and can lead to pain, stress, and potential skin tears from repeated application and removal. This is especially problematic in cases where wounds must be inspected multiple times per day. A bandage that enables non-invasive visualization of the wound site, while maintaining a secure barrier against bacteria and other contaminants over an extended period, would be highly advantageous. Research publications indicate non-adherence rates of 40% to 60% with respect to visual inspection due to the pain experienced during bandage changes.

In addition, bandages are often applied to body parts that move frequently or have complex geometries, such as limbs, joints, or curved areas. A major unmet need is the development of bandages that adhere securely to such surfaces while maintaining flexibility through advanced materials and adhesives. Traditional adhesive materials are often rigid, leading to discomfort for the patient or slippage of the bandage. In veterinary care, including equine, companion, exotic, and livestock medicine, this challenge is exacerbated by constant animal movement, making it difficult to maintain proper adhesion of the bandage over time.

Furthermore, many current bandage materials are not designed for prolonged use or repeated manipulation. This limitation is particularly problematic for chronic wound patients who require long-term dressings capable of withstanding multiple peel-and-reseal cycles. The durability of bandages under such conditions, particularly those that must support the reapplication of sterile treatments over extended periods, remains an area of ongoing research. Medical practitioners have identified a need for bandages that maintain their integrity after repeated adjustments, thereby ensuring that the wound remains covered and protected throughout an extended treatment period.

Infection control is another major concern in wound care, and bandages serve as the first line of defense against harmful pathogens. Current products often do not adequately seal the wound from external contaminants. In addition, frequent removal of bandages for inspection can introduce new infectious agents, thereby increasing the risk of contamination. Innovations that provide an improved seal while still allowing necessary treatment access could significantly reduce infection rates. Research has shown that minimizing wound exposure through advanced bandage designs can result in faster healing and fewer complications.

Cost is a significant factor in the accessibility of advanced wound care products. Nursing labor required to remove and reapply bandages multiple times per day substantially increases costs and stress for both patients and caregivers. For many patients, particularly in underdeveloped or rural areas, high-technology bandages may be prohibitively expensive. There is an unmet need for affordable bandaging solutions that incorporate advanced features, such as transparency, flexibility, and durability, without significantly increasing cost, while also reducing caregiver burden. The industry has been exploring materials and manufacturing processes that can bring these innovations to market at a lower price point, thereby expanding access to a broader patient base.

Increasing awareness of environmental sustainability has prompted interest in developing eco-friendly bandages. Comfort also remains a significant concern for both human and veterinary patients. Many bandages rely on adhesives that irritate or tear the skin upon removal, particularly in patients with delicate skin such as seniors, infants, and neonates. In addition, bandage materials must be breathable to prevent moisture build-up, which can lead to maceration of the skin surrounding the wound. There is a clear demand for bandages that are comfortable for extended wear, minimize the need for frequent disturbance, and provide the dual benefits of enhancing patient comfort and promoting healing.

Effective wound management requires continuous and accurate monitoring of physiological parameters to assess healing progress, detect complications such as infection, and guide treatment decisions. Traditional wound assessment often relies on visual inspection, periodic dressing changes, and laboratory tests, which are invasive, time-consuming, and provide only intermittent data. Existing sensor technologies for wound monitoring frequently lack transparency, flexibility, or the capability to measure multiple parameters simultaneously in a non-intrusive manner. There is a significant and growing need for wearable bandages containing sensor systems that provide intermittent or continuous real-time data on wound conditions without impeding visual inspection or requiring frequent removal.

Although the wound care market is substantial, there remains a lack of standardization in bandage products. Healthcare professionals often must choose among a variety of bandage types and brands, none of which may precisely meet the specific needs of a particular wound. Customization in bandage design, such as variations in size, adhesive strength, and configuration, could provide an effective solution to this problem. Bandages tailored to the size and nature of a wound, or adjustable in the field, would address a substantial unmet need in both human and veterinary medicine.

Despite numerous advancements in wound care technology, the industry continues to face significant unmet needs, particularly in the areas of wound monitoring, visibility, flexibility, infection control, and durability. Innovations in bandage design that address these challenges should substantially improve outcomes for patients and healthcare providers. As research in wound healing and material science progresses, there remains an opportunity to develop products that meet the specific demands of complex wound care, thereby improving patient care and overall quality of life.

### SUMMARY OF INVENTION

The present invention addresses multiple critical unmet needs in wound care, including the need for remote monitoring, visibility, flexibility, durability, infection prevention, affordability, environmental sustainability, labor savings, and patient comfort. Its architecture represents a significant advancement in wound care technology, offering a comprehensive solution to many of the challenges faced in both human and veterinary medicine.

In one aspect, the present invention provides a wound dressing system incorporating a peelable, transparent cover affixed to a flexible base layer. The cover enables wound inspection without full removal of the dressing and can be resealed multiple times, thereby reducing the risk of contamination, minimizing skin trauma, and supporting a stable healing environment.

In another aspect, the present invention incorporates a flexible, stretchable base material designed to conform to curved or moving body surfaces while maintaining secure adhesion. The peelable cover is resealable, allowing repeated access to the wound without compromising adhesive integrity.

In another aspect, the present invention integrates optional embedded sensors and communication circuits within the bandage system. These sensors are capable of monitoring wound conditions and skin parameters, and transmitting data to external devices for real-time or intermittent analysis.

In another aspect, the present invention employs materials and fastening mechanisms that enable the bandage to endure multiple peel-and-reseal cycles while maintaining durability and patient comfort.

In yet another aspect, the present invention supports infection control by providing a secure barrier against contaminants, even during repeated access to the wound.

In certain aspects, the present invention incorporates scalable, low-cost, and environmentally sustainable manufacturing processes, such as roll-to-roll production, to ensure affordability, high-volume availability, and reduced medical waste.

Thus, embodiments of the present invention overcome the unmet needs identified in the art by providing a wound dressing that delivers protection, visibility, durability, infection control, affordability, sustainability, and patient comfort.

### BRIEF DESCRIPTION OF THE DRAWINGS

For the present invention to be clearly understood and readily practiced, the present invention will be described in conjunction with the following figures, wherein like reference characters designate the same or similar elements, which figures are incorporated into and constitute a part of the specification, wherein:

FIG. 1 is a schematic view of one embodiment of the bandage system applied to an equine wound site, illustrating the peelable cover lifted for treatment access.

FIG. 2 is a schematic view of one embodiment of the bandage system applied to an equine limb, illustrating size configuration and peelable cover access.

FIG. 3 is a schematic view of one embodiment of the bandage system incorporating drainage and infusion catheters positioned at or near the wound site.

FIG. 4 is a schematic view of one embodiment of the bandage system applied to a human surgical wound, illustrating a dressing change procedure with the peelable cover.

FIG. 5 is a schematic view of one embodiment of the bandage system including a removable transparent cover with an integrated sensor array for remote wound monitoring.

FIG. 6 is a schematic view of one embodiment of the bandage system configured for a canine patient, illustrating a removable sensor-enabled cover and an adjustable strap accommodating loose skin and movement.

FIG. 7 is a schematic view of one embodiment of the bandage system configured as a pressure bandage for a canine patient, illustrating a removable transparent cover, strap system, drainage and infusion catheters, and peripheral sensor array.

It should be clear that the description of the embodiments and attached Figures set forth in this specification serves only for a better understanding of the invention, without limiting its scope. It should also be clear that a person skilled in the art, after reading the present specification could make adjustments or amendments to the attached Figures and above-described embodiments that would still be covered by the present invention.

### DETAILED DESCRIPTION OF THE INVENTION

The peelable bandage system described in this invention provides a comprehensive solution to multiple unmet needs in wound care. By allowing controlled access to the wound without compromising the protective environment, the invention reduces infection risk, facilitates monitoring, and supports the application of treatments. Its flexible and durable materials ensure that the bandage remains comfortable and effective during extended wear, making it suitable for both acute and chronic wound management in human and veterinary medicine. It should be appreciated that through this innovative design, the invention addresses critical challenges in wound care, improving patient outcomes and reducing caregiver burden.

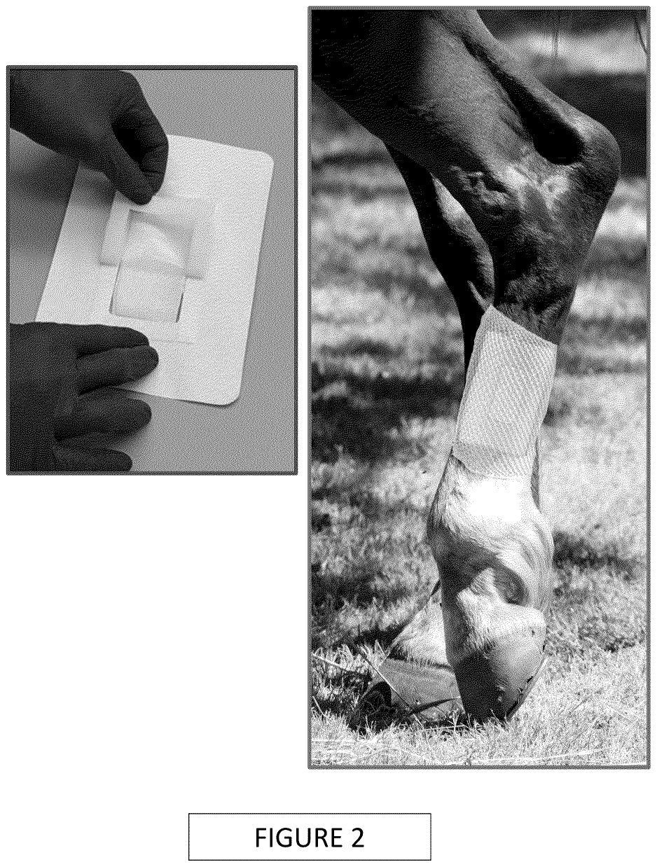

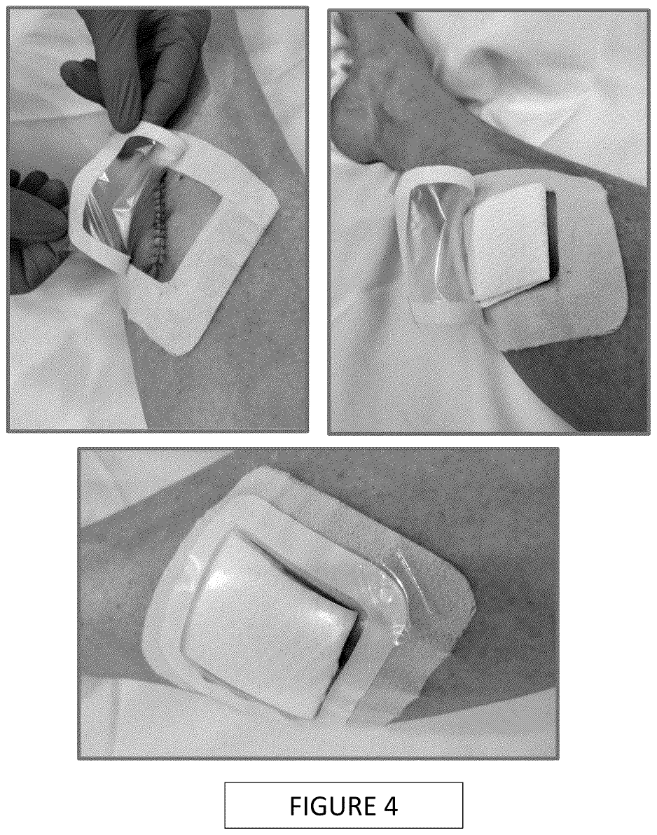

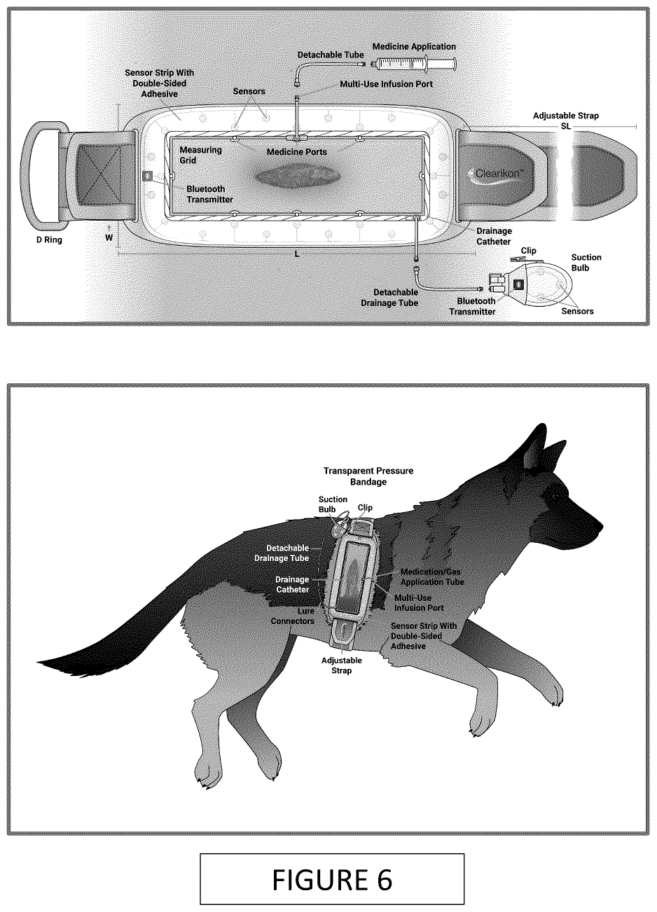

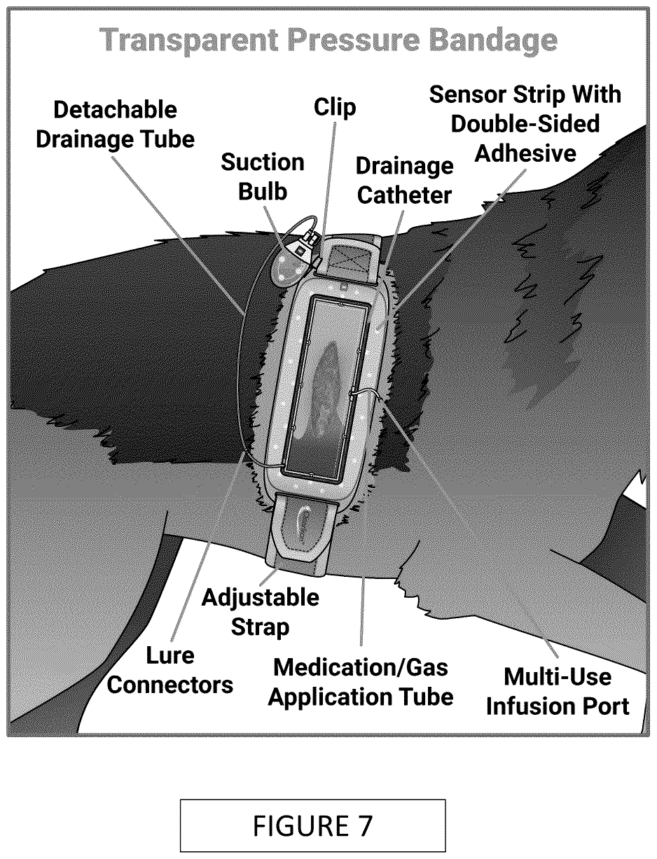

Certain embodiments of the present invention relate to a peelable bandage system configured to meet essential requirements of wound care, including preventing contamination, allowing visualization of the wound, facilitating drainage, and supporting extended wear with resealable features. This bandage system is particularly suited for human and veterinary applications as illustrated in FIG. 4 for human use, FIG. 2 for equine applications, and FIGS. 6 and 7 for canine applications, and addresses limitations of existing products, including the inability to monitor the wound without full removal, maintaining a closed environment for healing, and enabling treatments such as drainage, topical medications, or lavage.

Embodiments of the present invention include two principal components: a base layer and a peelable transparent cover. Together, these components form a secure, resealable environment that supports wound healing while reducing the need for frequent bandage changes. The peelable cover allows for repeated inspections and treatments without disturbing the wound bed, lowering the risk of contamination and improving usability for caregivers and medical professionals. In some embodiments, the peelable cover incorporates sensors and communication circuits configured to monitor wound conditions and transmit data to an external device.

The present invention offers several advantages in wound care, particularly addressing needs for visibility, infection control, durability, and patient comfort. In some embodiments, the transparent peelable cover provides continuous visualization of the wound, enabling monitoring and intervention without removing the bandage. Transparency and resealable access also support remote inspection via telemedicine. In other embodiments, the resealable attachment mechanism allows the cover to be opened and closed multiple times, reducing contamination risk while permitting treatment access. It is believed that the use of long-wear materials in the base layer and peelable cover maintains adhesion and performance over multiple peel-and-reseal cycles. In at least one embodiment, the flexible, stretchable base layer conforms to body contours, including joints and other high-mobility regions, reducing irritation and improving compliance, as illustrated in FIG. 4 for human applications, FIG. 2 for equine use, and FIGS. 6 and 7 for canine wound care.

### Definitions

The following detailed description is merely exemplary in nature and is in no way intended to limit the scope of the invention, its application, or uses, which may vary. The invention is described with relation to the non-limiting definitions and terminology included herein. These definitions and terminology are not designed to function as a limitation on the scope or practice of the invention, but are presented for illustrative and descriptive purposes only.

As used herein, a “peelable cover” refers to a transparent, removable layer affixed to the base layer via a resealable mechanism. The peelable cover permits visualization of the wound, enables repeated access for treatment, and reseals to maintain a closed wound environment that protects against contamination.

As used herein, a “base layer” refers to a flexible adhesive substrate configured to conform to patient skin and support the peelable cover. The base layer provides stability for the dressing, accommodates patient movement, and may incorporate antimicrobial agents, perforations, or embedded sensors.

As used herein, a “resealable mechanism” refers to adhesives, mechanical fasteners, or equivalent systems enabling repeated peel-and-reseal cycles without significant loss of adhesion. Non-limiting examples include medical-grade adhesives, hook-and-loop systems, and groove-based mechanical couplings.

As used herein, “telemedicine monitoring” refers to remote visualization or transmission of wound condition data to a healthcare provider. Such monitoring may be achieved through embedded sensors, wireless communication circuits, or direct visual inspection through the transparent peelable cover.

As used herein, “embedded sensors” refers to electronic devices integrated into the peelable cover or base layer that measure wound or skin parameters, including temperature, pH, exudate level, oxygenation, or biochemical markers.

As used herein, “scalable manufacturing” refers to processes such as die cutting, vapor deposition, or roll-to-roll fabrication that enable high-volume production of bandages while reducing cost and material waste.

As used herein, a “biodegradable material” refers to a material capable of breaking down naturally through biological processes after disposal, thereby reducing long-term environmental impact.

As used herein, a “recyclable material” refers to a material capable of being reprocessed and reused in manufacturing, thereby minimizing medical waste associated with wound dressings.

As used herein, “patient compliance” refers to the degree to which a patient follows prescribed treatment protocols, including maintaining the bandage in place, resealing it properly, and permitting timely inspection and treatment.

As used herein, a “bi-directional stretchable material” refers to a flexible substrate capable of elongating along at least two axes while returning to its original shape, thereby accommodating patient movement while preserving adhesion.

As used herein, a “resealable adhesive” refers to an adhesive layer formulated to retain adhesion properties through multiple peel-and-reseal cycles. Such adhesives are designed to secure the peelable cover to the base layer while allowing repeated removal and replacement.

As used herein, an “antimicrobial agent” refers to a substance that inhibits or destroys microorganisms, including but not limited to silver ions and chlorhexidine. These agents may be embedded within the base layer or applied as a coating to reduce infection risk.

As used herein, “exudate” refers to fluid emitted from a wound, composed of serum, blood, and cellular debris, the management of which is essential to prevent maceration and support healing.

As used herein, “perforations” or “drainage channels” refers to structural features within the base layer designed to direct wound exudate away from the wound site and into absorptive materials or external drainage systems.

As used herein, a “transparent peelable cover” refers to a removable protective layer affixed to the base layer that permits direct visualization of the wound and supports repeated resealing while maintaining a sterile environment.

As used herein, an “anti-fogging treatment” refers to a surface modification or coating designed to prevent condensation and maintain transparency of the peelable cover under humid or exudative conditions.

As used herein, a “resealable attachment mechanism” refers to adhesives, fasteners, or other systems that enable multiple peel-and-reseal cycles without significant reduction in sealing integrity or adhesion.

As used herein, a “UV-blocking layer” refers to a structural film or additive incorporated into the peelable cover to reduce ultraviolet radiation exposure that may otherwise damage healing tissue.

As used herein, a “tab” or “grip feature” refers to a structural extension of the cover that facilitates lifting without disturbing the adhesive bond between the cover and the base layer.

As used herein, “flexible electronics” refers to thin-film or printed circuits capable of conforming to non-planar surfaces, integrated into the peelable cover or base layer for sensing or communication.

As used herein, an “exudate sensor” refers to a sensor designed to detect and quantify wound fluid levels and characteristics, such as volume, conductivity, or viscosity.

As used herein, a “biosensor” refers to a sensor designed to detect specific biochemical markers, including protease activity, glucose, or bacterial metabolites, for assessment of wound status.

As used herein, a “periwound region” refers to the healthy skin immediately surrounding a wound site, the monitoring of which provides contextual information on healing and infection spread.

As used herein, a “low-power wireless protocol” refers to a communication standard, including Bluetooth Low Energy (BLE), Near-Field Communication (NFC), or Radio-Frequency Identification (RFID), optimized for short-range, energy-efficient data transmission.

As used herein, “energy harvesting” refers to the process of deriving power for sensors or circuits from ambient sources such as body heat, mechanical movement, or electromagnetic fields.

As used herein, a “data aggregation hub” refers to a local microcontroller or circuit within the base layer that collects data from embedded sensors and transmits it to an external device.

As used herein, an “access pathway” refers to a localized, resealable opening, port, conduit, or interface in the peelable cover that permits the introduction of treatments or devices while preserving the protective barrier.

As used herein, a “self-sealing aperture” or “grommet” refers to an elasticized port that closes upon removal of a catheter or device, thereby re-establishing a sterile seal.

As used herein, “mechanical lavage” refers to the delivery and evacuation of irrigants across the wound bed using a syringe, pump, or equivalent mechanism.

As used herein, a “drainage catheter” refers to a conduit positioned to remove wound exudate to an external suction reservoir or vacuum pump.

As used herein, a “suction reservoir” refers to a bulb, canister, or equivalent device configured to generate and maintain negative pressure for exudate collection.

As used herein, an “infusion catheter” refers to a conduit positioned to deliver therapeutic fluids or gases to the wound bed under controlled conditions.

As used herein, a “non-adherent primary dressing” refers to a wound-contact layer designed to minimize tissue adherence during dressing changes, placed beneath the peelable cover.

As used herein, an “energy-based therapy” refers to a treatment modality delivering thermal, optical, or acoustic energy, including heat, laser, or ultrasound, to the wound for therapeutic purposes.

In addition to the definitions above, it is to be understood that in instances where a range of values are provided that the range is intended to encompass not only the end point values of the range but also intermediate values of the range as explicitly being included within the range and varying by the last significant figure of the range. By way of example, a recited range from 1 to 4 is intended to include 1-2, 1-3, 2-4, 3-4, and 1-4.

### Generally

The present invention provides a bandage system that enables caregivers and patients to inspect, treat, and monitor wounds without requiring complete removal of the bandage. The system comprises a base layer configured to adhere to skin and a transparent peelable cover designed for repeated opening and resealing. Together, these components maintain a controlled wound environment that reduces contamination, supports extended wear, and allows efficient access for treatment.

In operation, the bandage system protects the wound in a closed environment while enabling visualization and treatment. The peelable cover lifts to provide access for medications, drainage, or inspection, and reseals without loss of adhesion. In certain embodiments, the base layer incorporates antimicrobial agents, the cover or base integrates sensors, or the system includes communication circuits to transmit wound condition data.

The invention incorporates features that support cost-effectiveness, scalability, and sustainability. In some embodiments, the bandage is manufactured using scalable processes such as die cutting, vapor deposition, or roll-to-roll production. These processes reduce manufacturing costs and enable high-volume production, thereby improving accessibility. It should be appreciated that durable materials reduce the frequency of replacement, lowering long-term costs for patients and caregivers.

In additional embodiments, the bandage system employs biodegradable or recyclable materials to reduce medical waste and environmental impact. The long-lasting durability of the invention further minimizes material consumption over the course of treatment.

Patient comfort is a central feature of the invention. The use of soft, flexible materials allows the bandage to conform to body contours, reducing irritation and enabling extended wear. It is believed that in veterinary applications, such comfort reduces distress to animals during treatment and improves compliance and treatment outcomes, as illustrated in FIG. 2 for equine wound care and in FIGS. 6 and 7 for canine applications employing straps and catheters to accommodate loose skin and frequent movement.

### Base Layer

The base layer operates as the foundational component of the wound dressing system. It adheres to the skin surrounding a wound and provides a stable surface for the transparent peelable cover. The base layer comprises a flexible, stretchable material that conforms to patient contours and accommodates body movement without losing adhesion. It should be appreciated that such flexibility allows the dressing to remain stable on complex or curved surfaces, including joints and areas subject to frequent motion.

In at least one embodiment, the base layer is constructed from a bi-directional stretchable material to provide flexibility and comfort. Suitable non-limiting examples include polyurethane foams, elastic woven fabrics, silicone-coated fabrics, and combinations thereof. These materials provide durability during extended wear while minimizing irritation.

In certain embodiments, the base layer employs a flexible adhesive formulated to accommodate modest movements while maintaining a secure fit for extended periods without frequent adjustments. An adhesive interface between the base layer and the peelable cover is resealable, enabling repeated opening and closing of the cover without compromising bond integrity. Without being bound by a particular theory, it is believed that embodiments employing this resealable adhesive structure remain effective and comfortable during prolonged wear, including on areas subject to frequent movement.

In certain embodiments, the base layer incorporates antimicrobial agents to provide continuous protection against contamination. Non-limiting examples include silver ions and chlorhexidine, both of which exhibit well-documented antimicrobial properties. These agents are embedded within the base layer material or applied as surface coatings. It is believed that by inhibiting bacterial infiltration at the wound site, the antimicrobial properties of silver and chlorhexidine reduce infection risk, particularly in chronic wounds or wounds in contaminated environments.

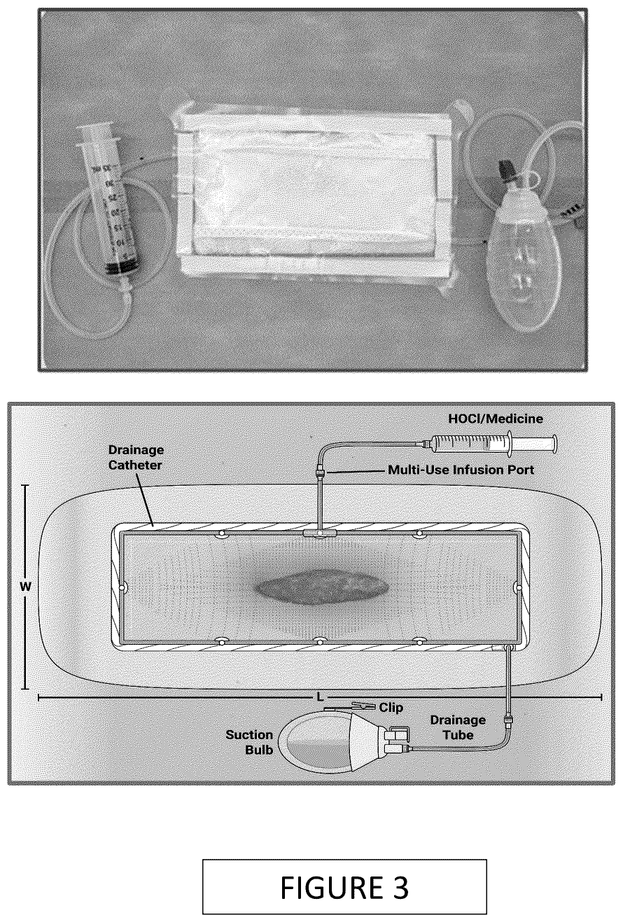

In at least one embodiment, the base layer includes perforations, drainage channels, or catheters to facilitate removal of wound exudate, as illustrated in FIGS. 3, 6, and 7. Materials such as porous polyurethane films or foamed polymers provide a breathable structure that supports fluid management while protecting the surrounding skin. Without being bound by a particular theory, maintaining a controlled moisture balance prevents maceration and supports healing.

In certain embodiments, the base layer integrates sensors configured to measure parameters of the surrounding skin region, including temperature, pH, and perspiration. It should be appreciated that such measurements provide actionable information for assessing healing progress and identifying early indicators of infection.

### Transparent Peelable Cover

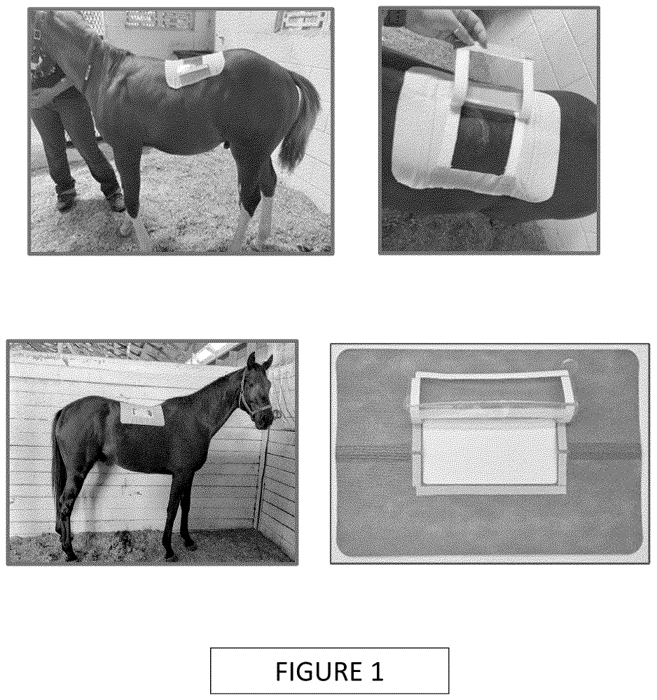

Embodiments of the present invention include a transparent peelable cover affixed to the base layer. In such embodiments, the combination of the transparent peelable cover affixed to the base layer, serves as the primary interface for inspection and treatment. The cover is designed for repeated removal and resealing without loss of adhesion, allowing caregivers to access the wound while the base layer remains securely in place. The transparency of the cover enables visualization without removal, thereby reducing the frequency of full bandage changes, lowering contamination risk, and supporting a stable healing environment. FIG. 1 illustrates an equine application of the transparent cover affixed to the base layer, FIG. 2 illustrates an equine leg-wound configuration, and FIG. 4 illustrates a human embodiment.

In at least one embodiment, the peelable cover is constructed from flexible, transparent materials selected to balance permeability, durability, and visibility. Non-limiting examples include thermoplastic polyurethane (TPU) films, polyethylene (PE) films, polyvinyl chloride (PVC), and silicone films. In some embodiments, anti-fogging agents are incorporated to maintain clear visualization over extended wear. It should be appreciated that flexibility and transparency together enable the cover to function as both a protective barrier and a visualization layer, as illustrated in FIGS. 1-2 for equine applications and FIG. 4 for human applications.

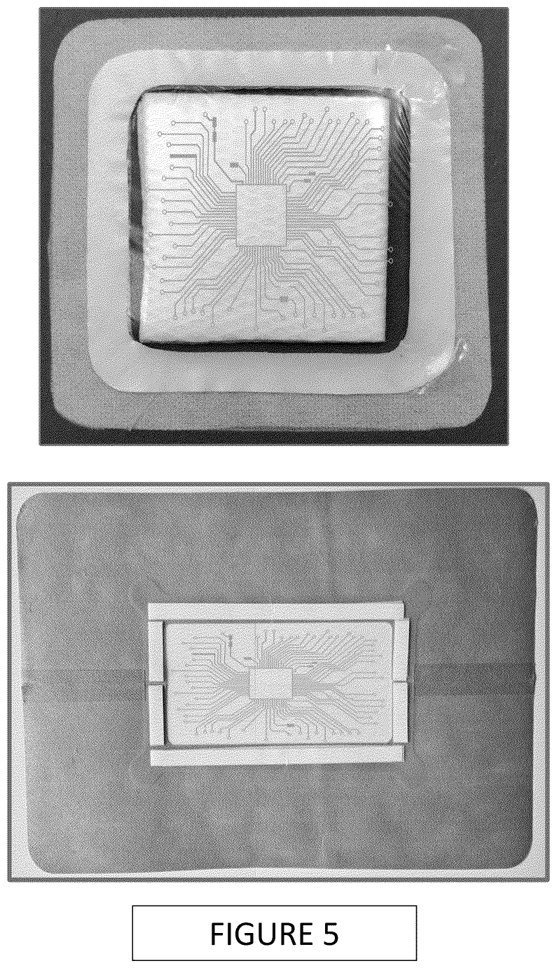

In certain embodiments, the transparent cover includes coatings or surface treatments that reduce condensation and maintain visibility of the wound site. Non-limiting examples of anti-fogging treatments include hydrophilic polymer coatings, surface etching, and chemical modification of the film surface. Without being bound by any particular theory, it is believed that these treatments distribute moisture evenly across the surface, preventing droplet formation and maintaining transparency. FIG. 5 depicts an embodiment of the cover including such surface treatments in conjunction with a sensor array.

In at least one embodiment, the cover incorporates a resealable attachment mechanism between the cover and the base layer. Non-limiting examples include medical-grade acrylic adhesives, silicone-based adhesives, hook-and-loop fasteners, and mating grooves. These attachment mechanisms retain adhesion properties through multiple peel-and-reseal cycles, including at least 14 cycles over a seven-day period. It should be appreciated that embodiments employing such resealable mechanisms allow for repeated wound access while maintaining protection and minimizing the need for full bandage replacement. FIGS. 1-2 and FIG. 4 depict representative use cases where resealable attachment enables repeated access.

In certain embodiments, the transparent peelable cover incorporates an additional layer that blocks ultraviolet (UV) radiation. The UV-blocking layer may be integrated within a multi-layer film structure, using materials such as polyethylene terephthalate (PET) films with UV absorbers. It is believed that UV protection prevents damage to healing tissue during exposure to sunlight, as relevant to equine applications shown in FIGS. 1-2 where wounds are exposed outdoors.

In additional embodiments, the transparent cover provides water resistance, protecting the wound from liquid contamination in external environments. FIGS. 6-7 illustrate veterinary applications where water resistance supports durability and hygiene in canine treatment settings.

In certain embodiments, the transparent cover includes a tab or grip feature designed to facilitate peeling without disturbing the adhesive interface, as shown in FIGS. 3 and 4. Non-limiting examples of suitable materials for the tab include silicone rubber and polyurethane foam. It should be appreciated that ergonomic tab structures improve ease of use for caregivers and reduce patient discomfort during wound access.

### Sensors and Communication Circuits

Embodiments of the inventive bandage system optionally integrates sensors and communication circuits within the transparent peelable cover, the base layer, or both. These components operate as a sensing and transmission platform, enabling continuous or intermittent monitoring of wound conditions without requiring removal of the bandage. Data collected by the sensors is wirelessly transmitted to an external device, such as a smartphone, tablet, or dedicated medical hub, for analysis, display, and alerts. FIG. 5 illustrates an embodiment of the transparent cover including integrated sensors and communication circuits.

In certain embodiments, the transparent peelable cover comprises a flexible, biocompatible substrate fabricated from materials such as medical-grade silicone, polyurethane, or transparent hydrogel. The transparency of the cover permits visual inspection of the wound, while its flexibility allows conformal contact with the wound bed or with an intermediate non-adherent dressing. In some embodiments, the cover is detachably secured to the base layer using resealable mechanisms such as hook-and-loop, snap-fit, magnetic coupling, or low-tack adhesives. FIG. 5 depicts an example of a sensor-enabled cover employing such attachment methods.

In at least one embodiment, the transparent peelable cover integrates an array of miniaturized sensors fabricated using flexible electronics techniques such as thin-film deposition or screen printing. Non-limiting examples of wound parameters monitored by such sensors include: i) temperature, measured by thermistor or thermocouple arrays, to identify inflammation or infection; ii) pH, measured by potentiometric or optical probes, to indicate healing progression or infection; iii) moisture and exudate characteristics, measured by impedance or conductive polymer sensors, to assess hydration balance; or iv) oxygen levels, measured by optical or electrochemical probes, to evaluate tissue viability. These arrays are illustrated in FIG. 5 as part of the integrated sensor system.

In additional embodiments, optional biosensors are incorporated to provide expanded diagnostic information. Non-limiting examples include protease activity sensors to detect elevated MMPs, glucose sensors for diabetic wound monitoring, bioimpedance sensors to assess edema, and bacterial load sensors for identifying infectious agents. It should be appreciated that such biosensors extend the scope of wound monitoring beyond basic vital parameters. FIG. 5 illustrates representative placement of such biosensors within the cover structure.

In certain embodiments, the base layer incorporates auxiliary sensors positioned around the peri wound region. Non-limiting examples include temperature sensors to establish baseline comparisons, hydration sensors to monitor the surrounding skin, pressure sensors to identify ulcer risk, and adhesion sensors to detect lifting or detachment. These measurements provide contextual data to complement wound-bed monitoring. FIG. 5 further shows integration of periwound sensors in combination with cover-based sensors.

Embodiments of the transparent peelable cover further includes communication circuitry configured for low-power wireless transmission of sensor data. Non-limiting examples of communication protocols include Bluetooth Low Energy (BLE), near-field communication (NFC), and radio-frequency identification (RFID). In at least one embodiment, BLE provides primary continuous data transmission, while NFC or RFID supports passive power harvesting or short-range data transfer. FIG. 5 depicts the integration of wireless communication circuits with the sensor array.

In at least one embodiment, the bandage system integrates a thin-film, flexible micro-battery sized for multiple days of continuous operation. In alternative embodiments, the cover includes passive RFID/NFC coils to harvest power from external readers, thereby enabling battery-free operation. In some embodiments, energy harvesting mechanisms such as thermoelectric or kinetic generators supplement or replace batteries to extend device life. Without being bound by any particular theory, it is believed that these hybrid power strategies improve usability by reducing the frequency of device replacement or recharging. FIG. 5 shows a representative integration of power components supporting sensor operation.

In some embodiments, the system incorporates onboard signal processing, including sensor data acquisition, amplification, filtering, analog-to-digital conversion, calibration, and packet formatting. Processed data is then wirelessly transmitted to the external device for display and alerting. In other embodiments, the base layer houses a low-power microcontroller and communication hub that aggregates data from the transparent cover before forwarding it to the external device. It should be appreciated that such distributed architectures provide flexibility in balancing device complexity, power efficiency, and cost. FIG. 5 illustrates an embodiment including local signal processing and communication functions.

A dedicated software application operating on an external device presents the collected data to clinicians or patients. Non-limiting features include real-time graphical displays, historical trend analysis, configurable alerts for abnormal parameters, and integration with electronic health records (EHRs). In certain embodiments, the software also logs visual observations of the wound, enabling correlation between sensor data and caregiver notes. FIG. 5 shows the data flow from the cover to an external device.

In at least one embodiment, the transparent peelable cover is detachable and replaceable, allowing sensor modules to be swapped while the base layer remains in place. This modularity supports long-term use by reducing replacement frequency and maintaining continuous data capture. FIG. 5 depicts such a detachable, sensor-enabled cover structure.

### Treatment Access and Delivery

The bandage system supports treatment access and delivery without requiring complete removal of the bandage. The transparent peelable cover lifts partially or fully and reseals to preserve a controlled wound environment between interventions. This structure enables wound inspection, topical application, irrigation, drainage, and infusion while maintaining barrier protection.

In certain embodiments, the transparent peelable cover includes localized access pathways configured to preserve the seal during and after treatment. Non-limiting examples include self-sealing apertures, resealable ports, or integrated conduits compatible with standard clinical connectors. It should be appreciated that such access pathways facilitate repeated treatment while limiting exposure to contaminants. FIGS. 6 and 7 illustrate veterinary applications incorporating access ports and conduits.

In at least one embodiment, topical agents are delivered through the access pathways or under a lifted cover and the cover is then resealed. Non-limiting examples of delivery forms include pre-medicated pads, gels, creams, solutions, and sprays applied directly to the wound bed or to a non-adherent primary dressing positioned over the wound.

In certain embodiments, mechanical lavage is performed through an access pathway using a syringe or pump to deliver and evacuate irrigants. The cover is resealed following lavage to re-establish the controlled wound environment. FIG. 3 illustrates an example of treatment access for lavage, while FIGS. 6 and 7 depict veterinary contexts where lavage may be combined with catheters and strap-based retention.

In at least one embodiment, drainage is established by positioning a drainage catheter through the access pathway and coupling the catheter to a suction reservoir or vacuum pump. Absorptive media, including gauze or other absorptive layers, is placed over the wound bed prior to resealing the cover. It should be appreciated that embodiments employing this configuration direct exudate away from the wound and reduce maceration. FIGS. 6 and 7 illustrate drainage catheter configurations in veterinary bandage systems.

In certain embodiments, infusion treatments are delivered through the access pathway using a catheter coupled to a syringe or infusion pump. Non-limiting examples include delivery of topical irrigants, buffered solutions, or oxygen-enriched gases for localized therapy. Without being bound by any particular theory, it is believed that localized infusion through a resealed cover maintains treatment concentration at the wound while limiting environmental exposure. FIGS. 6 and 7 depict infusion catheter arrangements positioned around veterinary wound sites.

The bandage system incorporates practices to support infection control during treatment. Sterile dressings, non-adherent primary layers, medications, and catheters are applied or replaced promptly, and the cover is resealed to restore barrier integrity. In certain embodiments, materials are sterilizable and supplied in sterile packaging suitable for field or clinical use. It should be appreciated that rapid exchange under a resealable cover reduces handling time and limits contamination risk.

In additional embodiments, the bandage system is used with adjunct topical therapies selected by the clinician. Non-limiting examples include (a) disinfectants (e.g., hypochlorous acid), (b) antimicrobial agents (e.g., antibiotics, antiseptics, antifungals, including silver sulfadiazine, iodine, chlorhexidine), (c) moistened dressings (e.g., hydrogels, alginates, hydrocolloids), (d) honey, (e) growth factors (e.g., PDGF, EGF), (f) stem cells, (g) enzymatic debriders, (h) topical oxygen therapy delivered via an access catheter, (i) temperature or humidity regulation via controlled gas flow, (j) energy-based therapies (e.g., heat, laser, ultrasound) applied through the transparent cover or directly under brief cover lift, and (k) microcurrent or TENS therapy via a conductive base layer and external device. FIGS. 6 and 7 depict examples where these adjunct therapies may be combined with catheter-based access.

In certain embodiments, at-home treatment is supported by supplying pre-packaged medicated pads and sterile accessories for scheduled replacement under the resealable cover. It should be appreciated that such embodiments reduce caregiver labor and improve adherence to prescribed wound-care protocols.

In at least one embodiment, access interfaces include luer-compatible fittings, check valves, or self-sealing grommets configured to maintain the barrier during catheter insertion and removal. The interfaces are positioned to avoid shear on the wound bed during patient movement and to permit secure dressing reclosure. FIGS. 6 and 7 illustrate such veterinary interfaces integrated with strap-based bandages.

Without being bound by theory, it is believed that embodiments providing rapid, sealed access for lavage, drainage, infusion, and topical therapy reduce contamination exposure, maintain moisture balance, and improve patient comfort relative to full bandage replacement workflows. FIGS. 1, 3, 4, 6, and 7 depict representative access operations under the resealable cover.

### EXAMPLES

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### Example 1

A peelable wound dressing system is applied to an equine limb wound. A flexible base layer adheres securely to the peri-wound skin, and a transparent peelable cover is affixed to the base layer by a resealable adhesive. The cover is lifted for inspection and then resealed without disturbing the base layer, enabling multiple peel-and-reseal cycles. The cover includes a tab feature to facilitate lifting and anti-fog treatment to maintain visibility. In this embodiment, the wound remains protected in a controlled environment, while repeated access is provided for topical treatments. FIGS. 1-2 illustrate representative equine applications.

### Example 2

In another embodiment, the peelable wound dressing system is adapted for canine veterinary use. A base layer with integrated straps conforms to the animal's torso, and a transparent peelable cover includes access ports for catheters. Through these ports, a drainage catheter is connected to a suction reservoir and an infusion catheter is coupled to a syringe pump for delivering therapeutic solutions. The resealable ports maintain barrier integrity after catheter removal, reducing contamination risk. FIGS. 6-7 illustrate exemplary canine applications with strap and catheter configurations.

### Example 3

In at least one embodiment, the transparent peelable cover incorporates a sensor network fabricated with flexible electronics. Sensors measure wound parameters such as temperature, pH, and moisture, while biosensors detect bacterial load and protease activity. Communication circuits transmit data via Bluetooth Low Energy to a paired smartphone application, which displays real-time graphs and alerts for abnormal conditions. In alternative embodiments, passive NFC coils harvest power during data collection, enabling battery-free operation. FIG. 5 illustrates an embodiment of the sensor-enabled cover.

### Example 4

In another embodiment, the bandage system provides sealed access for treatment delivery. Access pathways integrated into the peelable cover include self-sealing grommets and luer-compatible ports. Through these pathways, clinicians perform mechanical lavage, deliver oxygen therapy, or apply microcurrent stimulation using an external device. After each treatment, the pathways reseal, restoring the protective environment. It should be appreciated that this configuration reduces contamination exposure compared to full bandage replacement. FIG. 3 illustrates an embodiment incorporating drainage and infusion catheters.

### Example 5

A hybrid embodiment combines the features described in Examples 1-4. The base layer incorporates antimicrobial agents such as silver ions, while perforations facilitate fluid drainage. The peelable transparent cover includes anti-fogging and UV-blocking layers, sensor arrays for monitoring wound status, communication circuits for transmitting data, and access pathways for treatment delivery. Clinicians remotely track wound healing, receive alerts for infection indicators, and introduce therapies without disturbing the wound bed. FIGS. 1-7 collectively illustrate representative features of this hybrid embodiment.

### Other Embodiments

While at least one exemplary embodiment has been presented in the foregoing detailed description, it should be appreciated that a vast number of variations exist. For example, some uses do not require both ends of the apparatus to be secured to an object, and the apparatus may be hung or dangled from one end of an object. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient road map for implementing the exemplary embodiment or exemplary embodiments. It should be understood that various changes can be made in the function and arrangement of elements without departing from the scope as set forth in the appended claims and the legal equivalents thereof.

The foregoing description is illustrative of particular embodiments of the invention, but is not meant to be a limitation upon the practice thereof. The following claims, including all equivalents thereof, are intended to define the scope of the invention.

1. A peelable wound dressing system, the system comprising:
at least one base layer configured to adhere to at least one skin surface surrounding a wound; and
at least one peelable cover affixed to said at least one base layer by at least one resealable attachment mechanism;
wherein said at least one peelable cover is configured for repeated peel-and-reseal access to said wound without requiring removal of said at least one base layer.

2. The system of claim 1, wherein said at least one base layer comprises at least one flexible or stretchable material.

3. The system of claim 1, wherein said at least one base layer and said at least one peelable cover together form a closed environment for said wound.

4. The system of claim 1, wherein said at least one peelable cover further comprises at least one tab or grip feature to facilitate lifting during said repeated peel-and-reseal operations.

5. The system of claim 1, wherein said at least one peelable cover is transparent, enabling visualization of said wound without removal.

6. The system of claim 1, wherein said at least one base layer further comprises at least one antimicrobial agent selected from the group consisting of silver ions, chlorhexidine, iodine, antibiotics, antiseptics, antifungals, or combinations thereof.

7. The system of claim 1, wherein said at least one peelable cover further comprises at least one anti-fogging treatment configured to maintain clear visualization of said wound during extended wear.

8. The system of claim 1, wherein said at least one peelable cover further comprises at least one gas-permeable and water-resistant material configured to allow vapor exchange while preventing liquid contamination.

9. The system of claim 1, wherein said at least one resealable attachment mechanism maintains adhesion through at least 14 peel-and-reseal cycles over a period of at least 7 days.

10. The system of claim 1, wherein said at least one peelable cover comprises a plurality of layers, at least one of which is configured to block ultraviolet (UV) radiation.

11. The system of claim 1, wherein said at least one base layer further comprises at least one perforation, drainage channel, or catheter configured to facilitate removal of wound exudate.

12. The system of claim 1, wherein said at least one base layer further comprises at least one moisture-absorbing layer configured to regulate humidity within said closed environment.

13. The system of claim 1, wherein said at least one base layer comprises at least one bi-directional stretchable material configured to accommodate patient movement without loss of adhesion.

14. The system of claim 1, wherein said at least one peelable cover further comprises at least one access pathway selected from the group consisting of self-sealing apertures, resealable grommets, or luer-compatible ports.

15. The system of claim 14, wherein said at least one access pathway is configured to receive at least one drainage catheter coupled to at least one suction reservoir or vacuum pump.

16. The system of claim 14, wherein said at least one access pathway is configured to receive at least one infusion catheter coupled to at least one syringe or infusion pump.

17. The system of claim 14, wherein said at least one access pathway is configured to permit mechanical lavage of said wound bed.

18. The system of claim 1, wherein said at least one peelable cover and said at least one base layer together are configured to enable application of at least one topical agent selected from the group consisting of disinfectants, antimicrobial agents, hydrogels, alginates, hydrocolloids, honey, growth factors, stem cells, enzymatic debriders, oxygen therapies, energy-based therapies, or microcurrent therapies.

19. The system of claim 1, wherein said at least one peelable cover further comprises at least one sensor network integrated into said peelable cover to monitor wound parameters.

20. The system of claim 19, wherein said at least one sensor network is configured to measure at least one parameter selected from the group consisting of temperature, pH, moisture, oxygenation, protease activity, glucose level, bioimpedance, and bacterial load.

21.-50. (canceled)
